(19) United States
(12) Patent Application Publication
(10) Pub. No.: US 2004/0081968 A1
(43) Pub. Date: Apr. 29, 2004

(54) Method and mixture for curing autoimmune disease

(76) Inventor: Harvey Lang, Brooklyn, NY (US)

Correspondence Address: EDWARD N. GEWIRTZ, ESQ., BRONSTEIN, GEWIRTZ & GROSSMAN, LLC, SUITE 4600, 60 EAST 42ND STREET, NEW YORK, NY, 10165, US

(21) Appl. No.: 10/281,862

(22) Filed: Oct. 28, 2002

(51) Int. Cl. C12Q 1/68; A61K 31/70; A01N 43/04; C12P 21/06; A61K 39/12

(52) U.S. Cl. 435/6; 435/69.1; 514/44; 424/204.1

(57) ABSTRACT

Method and mixture for curing autoimmune disease. An Applied Kinesiology technique used to desensitize a patient to allergies is used to desensitize a patient to seven basic viruses. Desensitizing the patient to the seven basic viruses cures autoimmune disease. A homeopathic mixture of the seven basic viruses is used for optimal performance of the curative method.

### FIELD OF THE INVENTION

This invention relates to an Applied Kinesiology method for curing autoimmune disease and a homeopathic mixture of seven viruses that is used in a preferred embodiment of performance of the curative method.

### BACKGROUND

Autoimmune disease is defined as “disease in which the body produces disordered immunological response against itself. Normally the body's immune mechanisms are able to distinguish clearly between what is normal substance and what is foreign. In autoimmune diseases, this system becomes defective and produces antibodies against normal parts of the body to such an extent as to cause tissue injury.” (Taber's Cyclopedic Medical Dictionary) Common examples of diseases thought to be autoimmune disorders are systemic lupus erythematosis and many forms of arthritis. Even asthma, diabetes mellitus type 1 and certain cases of infertility are thought to be autoimmune in origin.

Prior art medical methods of treating autoimmune disease typically involve ongoing use of steroids, such as cortisone or prednisone. The invention disclosed herein, however, is a cure for autoimmune disease, using Applied Kinesiology techniques, that involves a quick, one-time, simple method.

The inventor found and earlier published that symptoms had improved in his patients with autoimmune diseases after he had desensitized those patients to either their own blood or to albumen (egg white) using an allergy desensitization technique. See “A Protocol For Dealing With Some Types of Autoimmune Disease,” Collected Papers of the Members of the International College of Applied Kinesiology, Volume I, Summer 1990-91, pp. 196-7, by Harvey Lang. The inventor further found and published therein that “An alternative approach, which probably is better and more specific, is to see if a particular virus(es) is associated with the above reaction and . . . desensitize to the virus(es), which will generally nullify the blood and/or albumen response.” Id. at 196.

Acting on his own advice, the inventor has developed a method for curing most autoimmune disease by desensitizing patients to seven basic viruses; Epstein-Barr, Coxsakie, Varicella, Herpes Simplex, Herpes Zoster, Herpes Progenitalis (sometimes called Herpes Simplex II) and Influenza (the “Seven Basic Viruses”).

Patients with autoimmune problems will show sensitivity to one of the Seven Basic Viruses. This sensitivity may be detected by use of well known Applied Kinesiology methods for testing for the temporary weakening of a patient's muscles. When a patient is exposed to a substance that the patient is sensitive to, which in the case of an autoimmune patient is one of the Seven Basic Viruses, the patient's muscles will be temporarily weakened.

The muscle weakness test may involve simply having a patient hold a homeopathic vial containing one of the Seven Basic Viruses in the patient's hand, while the clinician then manually tests the patient's muscle strength. For example, the clinician can test for changes in the patient's ability to hold up the patient's elbow while the clinician tries to push the patient's elbow downward. The clinician can more easily press down the patient's elbow when the patient holds in his/her hand a homeopathic vial containing that one of the Seven Basic Viruses that the patient is sensitive to.

Certain symptoms are generally characteristic of each specific virus. For example, patients complaining of asthma, eczema or various arthritides, such as rheumatoid arthritis, are usually sensitive to Varicella (Varicella is sometimes equated with Herpes Zoster viruses by many practioners. However they act differently with respect to this method. Varicella virus is cultured from persons with Chicken Pox. Herpes Zoster is cultured from persons with Shingles). Patients who are sensitive to Varicella show a 100% correlation with sensitivity to nightshade vegetables (eggplant, green and red peppers, tomatoes, tobacco and potatoes). Herpes Zoster sensitivity presents with a milder form, but similar to the above symptoms of Varicella without the nightshade problem. Influenza is associated with diabetes mellitus type 1. Patients with gastrointestinal disorders, such as irritable bowel syndrome or colitis (including ulcerative and Crohn's), demonstrate sensitivity to Herpes Simplex. Chronic Fatigue Syndrome has been often associated with either Epstein-Barr or Coxsakie also showing allergy to egg yellow with at least minor liver problems. Patients presenting with psoriasis often show sensitivity to Herpes Simplex Type 2, sometimes called Herpes Progenitalis or Herpes Genitalis.

The inventor has found that correction of the above autoimmune diseases can be accomplished by desensitizing a patient to the Seven Basic Viruses. For the method to be effective, one must desensitize the patient to all seven different virus syndromes because, as a matter of fact, correcting one virus syndrome causes a second, etc. until all seven are corrected.

Through further research, the inventor found that if all the Seven Basic Viruses were mixed together in one homeopathic vial (the “Combination 7 Vial”), a patient could be desensitized to all of the Seven Basic Viruses by having the patient hold the Combination 7 Vial, and by then performing the allergy type desensitization method on the patient seven consecutive times.

### SUMMARY OF THE INVENTION

To cure a patient from autoimmune disease, an allergy desensitization technique is used to desensitize a patient to each of the Seven Basic Viruses; Epstein-Barr, Coxsakie, Varicella, Herpes Simplex, Herpes Zoster, Influenza and Herpes Simplex Type 2. The allergy desensitization technique is performed as if the patient were allergic to each of the Seven Basic Viruses. When the Seven Basic Viruses are combined into one homeopathic vial, the method for curing autoimmune disease is performed more easily.

### DESCRIPTION OF THE PREFERRED EMBODIMENT

The curative method may be implemented by use of any of a number of allergy desensitization techniques. The preferred allergy desensitization technique known to the inventor for implementing the method for curing autoimmune disease is a laser desensitization technique. This technique, in a slightly different form, was originally developed by Dr. Michael Lebowitz. The technique as originally developed by Dr. Lebowitz involved having a patient put an allergin in his mouth or on his philtrum (above the upper lip), and then applying a laser to four points on the patient's head. The clinician would then test the patient's muscle strength. The clinician would next place a drop of the patient's own blood on the patient's tongue or philtrum, and again apply the laser on the four points.

The inventor found as a practical matter that his patients did not like having blood drawn from them, even a small drop. The inventor thus improved upon Dr. Lebowitz'technique by using the patient's saliva placed on the patient's philtrum, rather than the patient's blood. Some of the patient's saliva is collected on a cotton applicator (commonly know as a Q-tip), and the cotton applicator is then placed on the patient's philtrum (the groove just above the center of the upper lip) while the laser is applied to the four points.

The laser should be a red laser of wavelength 635 nm, 650 nm or 675 nm. The patient holds in his/her hand a closed homeopathic vial containing the virus which the patient is being desensitized to. While the patient holds the vial in his/her hand, a laser is applied at four points: just above the two eyebrows, at the anterior fontanel (also called governing vessel 21) and at the posterior fontanel (also called governing vessel 20). The patient's muscle strength is then tested. Next, a cotton applicator, dampened by the patient's saliva, is placed on the patient's philtrum, and the laser is again applied to the four points: just above the two eyebrows, at the anterior fontanel (also called governing vessel 21) and at the posterior fontanel (also called governing vessel 20).

An alternative allergy desensitization technique that may be used involves applying pressure to a patient's eyes. This technique is fully described in “The Thirty-Second Allergy Cure,” Collected Papers of the Members of the International College of Applied Kinesiology—U.S.A., Volume I, 1988-89, by Harvey Lang. As will be obvious to those skilled in the art, other desensitization techniques may also be used in place of the laser technique, as part of the method for curing autoimmune disease.

As a diagnostic matter, a clinician who is going to apply the disclosed method for curing autoimmune disease should first test the patient for sensitivity to each of the Seven Basic Viruses separately. While the testing of each of the Seven Basic Viruses separately is not necessary for performance of the autoimmune cure which is the object of the invention, it would nonetheless be prudent for a clinician to know the patients' sensitivities to the individual basic viruses, in order to analyze the correlation between symptoms the patient has complained of and the effect of the treatment for autoimmune disease. The patient holds a homeopathic vial of one of the Seven Basic Viruses in his/her hand and the clinician tests a previously competent muscle. For example, the patient is asked to hold his/her arm up while bent at the elbow. The clinician simultaneously presses down on the patient's elbow to test the patient's deltoid muscle strength. If the patient is sensitive to one or more of the viruses, that patient's deltoid muscle will be weakened when the vial of the virus is being held in the patient's hand. The test can likewise be performed using competent muscles other than the deltoid muscle.

A patient should first be desensitized to that one of the Seven Basic Viruses that the patient is sensitive to, i.e. the one that causes the muscle weakness. When all of the Seven Basic Viruses are mixed together in one homeopathic vial, the curative method may thus be performed most easily. This is so because the inventor has found that the patient's body automatically selects which of the Seven Basic Viruses the patient is being desensitized to on each of the seven performances of the allergy desensitization type technique. The body automatically selects the virus to which the patient is most sensitive in the first instance, selecting each time a different of the viruses from the vial to desensitize the patient to until the patient has been desensitized to all of the Seven Basic Viruses. The order in which the patient is being desensitized is not needed by the clinician, who need only have the patient desensitized seven times while holding the one vial containing all seven basic viruses. As stated above, it is necessary to desensitize a patient to all of the Seven Basic Viruses, because desensitization to one will cause sensitivity to another, until the patient has been desensitized to all of the Seven Basic Viruses. While live virus cultures could also be used, rather than a homeopathic vial of the virus, it would be very dangerous to use live viral culture because of the risk of contagiousness to the patient and clinician.

There are some causative conditions that have to also be dealt with because they effect the permanence or relative permanence of the cure. One causative condition is parasites, which in the inventor's opinion is one of the major triggers for autoimmune problems. Techniques for ridding a patient of parasites are well known in the fields of traditional and alternative medicine.

I claim:

1. A mixture for use in curing a patient from autoimmune disease comprising:
a. Epstein-Barr virus;
b. Coxsakie virus;
c. Varicella virus;
d. Herpes Simplex virus;
e. Herpes Zoster virus;
f. Influenza virus; and
g. Herpes Simplex Type 2 virus.

2. The mixture of claim 1 wherein the seven viruses are contained in a homeopathic vial.

3. The mixture of claim 1 wherein the mixture is a homeopathic mixture.

4. A method for curing autoimmune disease which comprises:
a clinician desensitizing a patient to Epstein-Barr virus, Coxsakie virus, Varicella virus, Herpes Simplex virus, Herpes Zoster virus, Influenza virus and Herpes Simplex Type 2 virus.

5. The method of claim 4 wherein the clinician desensitizes the patient to the viruses using an allergy desensitization technique.

6. The method of claim 5 wherein the allergy desensitization technique comprises the steps of:
a. the patient holding a homeopathic vial of the viruses,
b. the clinician applying a laser above the center of the patient's right eyebrow, above the center of the patient's left eyebrow, at the patient's anterior fontenel, and at the patient's posterior fontenel;
c. the clinician testing the patient's muscle strength;
d. the clinician placing a cotton applicator containing the patient's saliva on the patient's philtrum while the clinician applies a laser above the center of the patient's right eyebrow, above the center of the patient's left eyebrow, at the patient's anterior fontenel, and at the patient's posterior fontenel, and
e. the clinician repeating steps b, c and d six times.

7. The method of claim 6 wherein the laser is of wavelength 635 nm.

8. The method of claim 6 wherein the laser if of wavelength 650 nm.

9. The method of claim 6 wherein the laser if of wavelength 675 nm.
